(19) United States
(12) Patent Application Publication
(10) Pub. No.: US 2024/0238450 A1
(43) Pub. Date: Jul. 18, 2024

(54) ELECTROPORATION GENE THERAPY FOR TISSUE BARRIERS

(71) Applicant: Cochlear Limited, Macquarie University, NSW (AU)

(72) Inventor: Wolfram Frederik DUECK, Berlin (DE)

(21) Appl. No.: 18/559,407

(22) PCT Filed: Apr. 20, 2022
(86) PCT No.: PCT/IB2022/053690

§ 371 Date: Nov. 7, 2023

Related U.S. Application Data
(60) Provisional application No. 63/187,564, filed on May 12, 2021.

(51) Int. Cl. A61K 48/00; A61K 9/00; A61N 1/32

(52) U.S. Cl. CPC A61K 48/0083; A61K 9/0009; A61N 1/327

(57) ABSTRACT

Presented herein are techniques to deliver therapeutic substances to a fluidically-sealed chamber within the body of a recipient without compromising the tissue barrier. More specifically, a genetic treatment material is introduced proximate to a tissue barrier, such as the blood-labyrinth barrier, in a recipient. The cells of the tissue barrier are electroporated via implanted electrodes to transfer a least a portion of the genetic treatment material into the cells of the tissue barrier.

### BACKGROUND

### Field of the Invention

The present invention relates generally to electroporation gene therapy for tissue barriers.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: introducing genetic treatment material proximate to a blood-labyrinth barrier (BLB) in a recipient; and electroporating cells of the blood-labyrinth barrier to transfer a least a portion of the genetic treatment material into the cells of blood-labyrinth barrier.

In another aspect, a method is provided. The method comprises: positioning at least one electrode within a first spatial region adjacent to a blood-labyrinth barrier, the blood-labyrinth barrier disposed substantially between the first spatial region and a second spatial region in a body of a recipient; delivering deoxyribonucleic acid (DNA) as part of non-viral vectors encoding one or more therapeutics into the first spatial region; and electroporating cells of the blood-labyrinth barrier via the at least one electrode to cause the therapeutic to be expressed by the cells of the blood-labyrinth barrier into the second spatial region.

In another aspect, a method is provided. The method comprises: accessing a tissue barrier associated with a fluidically-sealed chamber in a body of a recipient; positioning a plurality of electrodes proximate to the tissue barrier; delivering one or more of deoxyribonucleic acid (DNA) vectors or Ribonucleic acid (RNA) vectors encoding one or more therapeutics proximate to the tissue barrier; and electroporating cells of the tissue barrier with the plurality of electrodes to cause the one or more therapeutics to be expressed by the cells of the tissue barrier.

### BRIEF DESCRIPTION OF THE DRAWINGS

Embodiments of the present invention are described herein in conjunction with the accompanying drawings, in which:

FIG. 1 is a schematic diagram illustrating the ear of a recipient, with which certain embodiments presented herein can be implemented;

FIG. 2 is a schematic diagram illustrating a gene therapy electroporation system, in accordance with certain embodiments presented herein;

FIGS. 3A and 3B are schematic diagram illustrating the gene therapy electroporation system of FIG. 2 used with the ear of the recipient of FIG. 1;

FIGS. 4A and 4B are schematic diagram illustrating an alternative gene therapy electroporation system, in accordance certain embodiments presented herein;

FIG. 5 is a schematic diagram illustrating another gene therapy electroporation system, in accordance certain embodiments presented herein;

FIG. 6 is a flowchart illustrating an example method, in accordance with certain embodiments presented herein;

FIG. 7 is a flowchart illustrating another example method, in accordance with certain embodiments presented herein; and

FIG. 8 is a flowchart illustrating a further example method, in accordance with certain embodiments presented herein.

### DETAILED DESCRIPTION

A growing area of research and development relates to the use of pharmaceutical compounds, biological substances, bioactive substances, etc., including pharmaceutical agents/active pharmaceutical ingredients (APIs) including commercially available versions of the same, genes, nucleases, endonucleases, nucleic and ribonucleic acids such as messenger RNA (mRNA), siRNA and miRNA, DNA vectors, oligonucleotides, antisense polynucleotides, peptides, polypeptides, proteins including binding proteins, anti-oxidants, and signalling compounds that promote recovery and resolution, other chemicals, ions, and molecules used to modulate inflammation within the body of individual. A person of ordinary skill in the art will appreciate that each of these substances can be generated by methods known in the art. Any one or more of the above listed molecules and compounds may be coupled to form a conjugated molecule.

Conjugated molecules are, as the name suggests, molecules linked together to form a complex, which can be administered for treating a wide variety of disorders and diseases. These molecules are characterized by having a cell-permeable (or -penetrating) component that facilitates delivery of another, linked component, a molecule or compound with biological activity, to intracellular or intranuclear sites of action where they may elicit a variety of effects, including, e.g., among other things, the regulation of gene expression through interference with post-transcription processes. Some conjugated molecules have been used for facilitated transport of bioactive molecules across the blood labyrinth and blood brain barriers, and similarly may be used for applications at the blood retinal barrier, among others. Numerous types of vectors can be used to deliver and express one or more therapeutic molecules in targeted cells.

Small interfering RNA (siRNA) molecules are a prime example of an active molecule that can be delivered, as a complex with a vector, to a target cell. While the high molecular weight and negative charge of double-stranded siRNA molecules would prevent them from crossing the blood labyrinth barriers, siRNA molecules have been coupled to vectors to facilitate transport to sites difficult to access. In one instance, labeled siRNA was delivered to inner ear cells by coupling it to a non-viral vector and injecting it into the middle ear where it permeated the round window member and gained access to inner ear cells of various kinds (Qi et al., 2014). These conjugated molecules have the potential to modulate the permeability and potentially several other properties of the brain-labyrinth barrier as well. The siRNA molecules have been used successfully to modulate the brain-labyrinth barrier by interfering with the production of connexin 43, an important protein constituent of the tight junctions between endothelial cells of the brain-labyrinth barrier; connexin 43 is involved with the regulation of brain-labyrinth barrier integrity (Zhang et al., 2020). The siRNA molecules further have been used in intracochlear gene therapy research (in mice) to target allele suppression to slow the progression of hearing loss (Yoshimura et al., 2019).

In certain aspects, inhibition of, e.g., expression of inflammatory cytokines can be achieved by administration of pharmaceutical formulations comprising inhibitory nucleic acids (e.g., dsRNAs, siRNAs, antisense oligonucleotides, etc.) directed to inhibit cytokine expression or activity. In some embodiments, pharmaceutical formulations comprise siRNA molecules coupled with transporter proteins or other molecules to facilitate entry into cells and nuclei, including those of the cells comprising the brain-labyrinth barrier. In one embodiment the transporter protein (coupled to a siRNA molecule) is one that is recognized by a brain-labyrinth barrier cell surface receptor, enabling endocytosis. In other embodiments one or more siRNA molecules comprise the pharmaceutical formulations. In some method embodiments, such solutions are prepared and administered to modulate permeability and possibly other properties of barrier tissues.

In some embodiments of solutions to be administered according the methods disclosed, a siRNA molecule may be coupled with a transport molecule for targeting and suppressing overexpression. In still other embodiments pharmaceutical formulations comprising siRNA molecules are administered target the expression of immune system actors such as TNF-α, IL-1β and other cytokines responsible for the immune response to injury or irritation, i.e., those having anti-pneumolysin activity. In some pharmaceutical formulation embodiments, the siRNA molecules may be protected from degradation by being packaged in known non-viral nano-particle-based carrier systems, or encased in polymers, silica, porous silicone, or lipids, for example (Kim et al., 2019). Pharmaceutical formulation embodiments may be combined with other such embodiments to be implemented in one or more disclosed method embodiments to modulate tissue barrier properties, including permeability.

Further, in some embodiments known gene editing technology may be used to excise or replace sections of genes that, e.g., encode regulators or cytokine availability at the brain-labyrinth barrier and surrounding tissues. For example, in some embodiments, gene editing strategies employing the various technologies known in the art, including but not limited to the CRISPR/cas9 system, among others, are used to correct genetic disorders to the extent such disorders manifest as permeable brain-labyrinth barrier (and other tissue barrier) malfunctions. A person of ordinary skill in the art would appreciate that other gene editing technologies known in the art may be used in such embodiments, and other Cas or other enzymes, proteins or peptides may be functional in the Cas9 or similar role. Gene editing methods known in the art can be performed upon the cells of a subject in vivo (or ex vivo and then administered as a component of a pharmaceutical formulation in the disclosed embodiments). Stem cell therapies may further be used to generate components of pharmaceutical formulation embodiments.

In some embodiments of pharmaceutical formulations, those comprising one or more compounds having or producing anti-caveolin-1 activity and/or anti-pneumolysin activity are effective for modulating the permeability and possibly other properties to improve brain-labyrinth barrier integrity. In other method and pharmaceutical formulation embodiments, combinations comprising any two or more of corticosteroids, vasoconstrictors and compounds having or producing anti-caveolin-1 activity and/or anti-pneumolysin activity in any form may be administered simultaneously or in serial by any single or combination of modes of administration to modulate the permeability of the brain-labyrinth barrier. Such embodiments may be used as a part of treatment regimens involving monitoring and preventing inflammation due to the increased expression of cytokines at or near the brain-labyrinth barrier and resulting loss of barrier integrity.

The body of an animal, including the body of a human recipient (“recipient”), includes a number of different fluidically-sealed chambers (e.g., cavities or enclosed areas in which bodily fluids are sealed off from other areas through semi-permeable tissue barriers). For example, sensitive tissues in the body of a recipient, such as the brain, the ear, the eye, etc. are protected from the normal circulation by fluidic tissue barriers. In particular, the brain is surrounded by the blood-brain barrier (BBB), the inner ear (including the cochlea and the vestibular system) are surrounded by the blood-labyrinth barrier (also referred to the BLB herein), the eye retina is surrounded by the blood-ocular barrier (BOB), which includes the blood-aqueous barrier (BAB) and the blood-retinal barrier (BRB), and so on. The round window and/or the oval window membranes, in particular, form part of blood-labyrinth barrier and are associated with a fluidically-sealed cochlea of a recipient.

As noted, it can be advantageous to deliver therapeutic substances to the body of a recipient. However, using conventional techniques, it is difficult to deliver therapeutic substances to fluidically-scaled chambers within the body of a recipient without compromising the near-term or long-term structural and functional integrity of the associated fluidic tissue barrier (e.g., without damaging the barrier that either will allow the fluid within the chamber to leak out and/or allow toxins, bacteria, viruses, large molecules and compounds to enter into the chamber). A tissue barrier with a compromised structural and functional integrity (i.e., that is “leaky”) is indicative of a “barrier disorder,” and may lead to malfunction of the tissue(s) and/or organ(s) that it is intended to protect. Moreover, fluidically-sealed chambers are often difficult for a surgeon or other medical practitioner to access.

Presented herein are techniques to deliver therapeutic substances to a fluidically-sealed chamber within the body of a recipient without compromising the tissue barrier (e.g., without the need to physically open a path to the inner ear). More specifically, and as described further below, a genetic treatment material is introduced proximate to a tissue barrier, such as the blood-labyrinth barrier, in a recipient. The cells of the tissue barrier are electroporated via electrodes to transfer a least a portion of the genetic treatment material into the cells of the tissue barrier. The electrodes can be proximity to, adjacent to, or abutting an outer surface of the tissue barrier and/or can be penetrating electrodes that only partially perforate the tissue barrier.

The genetic treatment material, following transfer into the tissue barrier cells, causes the tissue barrier cells to express (e.g., produce and release) one or more desired therapeutic substances (e.g., one or more therapeutic proteins). Since the cells of the tissue barrier are in contact with the body fluid within the fluidically-sealed chamber, the one or more desired therapeutic substances are released into the chamber without a need to be able to permeate the tissue barrier, which in turn may allow larger and charged molecules to be delivered without risk of causing a barrier disorder or other issues.

The techniques presented herein are sometimes referred to as “electroporation gene therapy techniques.” That is, the techniques presented herein involve transferring genetic material to cells. Functional copies of genes can be transferred to cells to replace faulty genes, or genes can be edited within the cells in vivo or ex vivo to correct a malfunction, or can be removed from the cell entirely, or bound to other nucleic acids (such as siRNA) to block production of a particular protein.

Merely for ease of description, the electroporation gene therapy techniques presented herein will primarily be described with reference to the delivery of therapeutic substances to a specific fluidically-sealed chamber of a recipient, namely the cochlea of a recipient behind the round window. However, it is to be appreciated that the electroporation gene therapy techniques presented herein can be used to deliver therapeutic substances to other fluidically-sealed chambers within the body of a recipient behind other tissue barriers.

The electroporation gene therapy techniques presented herein can be used alone or in combination with several types of implantable medical devices. For example, the techniques presented herein may be implemented with auditory prostheses, such as middle ear auditory prostheses, bone conduction devices, direct acoustic stimulators, electro-acoustic prostheses, auditory brain stimulators, cochlear implants, combinations or variations thereof, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

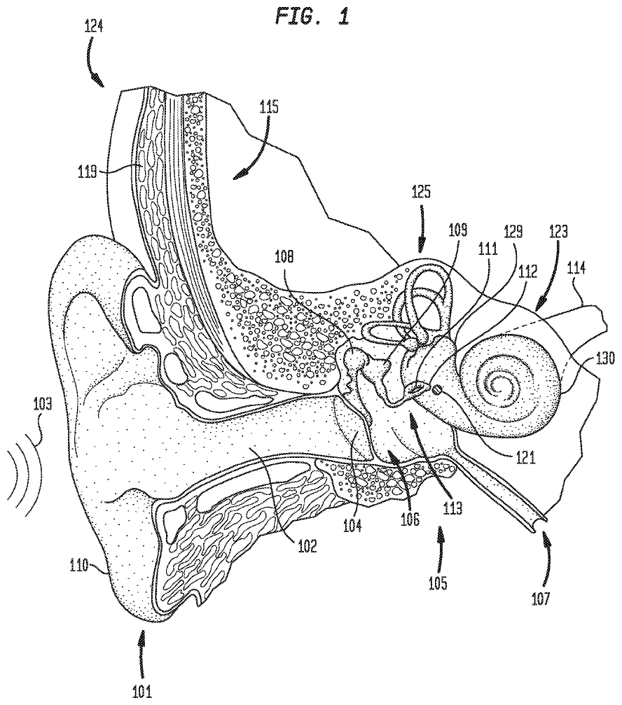

As noted, the electroporation gene therapy are primarily described herein with reference to the delivery of therapeutic substances to the cochlea of a recipient. Before describing details of the electroporation gene therapy techniques, basic structures of the ear of a recipient, including a cochlea with which the electroporation gene therapy techniques may be used, are first described below with reference to FIG. 1.

FIG. 1 illustrates that a recipient's ear normally comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121, through the bones of the middle ear 105. The bones of the middle ear 105 comprise the malleus 108, the incus 109 and the stapes 111, collectively referred to as the ossicles 106. The ossicles 106 are positioned in the middle ear cavity 113 and serve to filter and amplify the sound wave 103, causing oval window 112 to articulate (vibrate) in response to the vibration of tympanic membrane 104. This vibration of the oval window 112 sets up waves of fluid motion of the perilymph within cochlea 130. Such fluid motion, in turn, activates tiny hair cells (not shown) inside of cochlea 130. Activation of the hair cells causes appropriate nerve impulses to be generated and transferred through the spiral ganglion cells (not shown) and auditory nerve 114 to the brain (also not shown) where they are perceived as sound

The human skull is formed from a number of different bones that support various anatomical features. Illustrated in FIG. 1 is the temporal bone 115 which is situated at the side and base of the recipient's skull 124 (covered by a portion of the recipient's skin/muscle/fat, collectively referred to herein as tissue 119). Also shown in FIG. 1 is the bony labyrinth 123, which is a rigid bony structure surrounding the inner ear 107. The bony labyrinth 123 consists of the semicircular canals 125, the vestibule 129, and the cochlea 130, which are chambers/cavities hollowed out of the substance of the bone, and lined by periosteum. The semicircular canals 125, the vestibule 129, and the cochlea 130 each include a corresponding portion of the membranous labyrinth (e.g., the vestibule contains the utricle and saccule, each semicircular canal contains a semicircular duct, and the cochlea contains the cochlear duct). The membranous labyrinth is filled with a fluid called endolymph and surrounding the membranous labyrinth and filling the remaining space in the bony labyrinth 123 is the perilymph.

The bony labyrinth 123 includes two membrane-covered openings, the oval window 112 (oval window membrane) and the round window 121 (round window membrane). As noted, the oval window 112 vibrates in response to the vibration of tympanic membrane 104. The cochlea 130 is a closed, fluid-filled chamber such that the round window 121 vibrates with opposite phase to the vibrations entering the cochlea 130 through the oval window 112. As such, the round window 121 allows the perilymph in the cochlea 130 to move (in response to vibration at the oval window 112), which in turn ensures that hair cells of the basilar membrane will be stimulated and that audition will occur. The oval window 112 (oval window membrane) and the round window 121 (round window membrane) are tissue barriers that maintain the fluidic seal of the cochlea 130.

As noted above, delivery of therapeutic substances, such as active pharmaceutical ingredients (API), to the inner ear 107 (e.g., cochlea 130) remains a challenge due to the inner ear being behind the blood-labyrinth barrier (BLB). Systemically circulating therapeutic substances only pass the blood-labyrinth barrier in a fraction of the blood concentration which requires very high dosing with the increased risk of side effects to be effective in the inner ear 107. Another conventional approach is to deliver the therapeutic substances directly into the inner ear 107. Although this direct delivery is highly effective in delivering a therapeutic concentration, such approaches risk mechanical damage to the inner ear 107, which can result in hearing loss and increased risk of infection. That is, since the cochlea 130 is a fluidically-scaled chamber, maintaining the fluidic seal thereof is important to, for example, maintain residual hearing in the cochlea 130, ensure the integrity of the blood-labyrinth barrier, etc. Direct delivery approaches can comprise the fluidic seal and/or cause other damage.

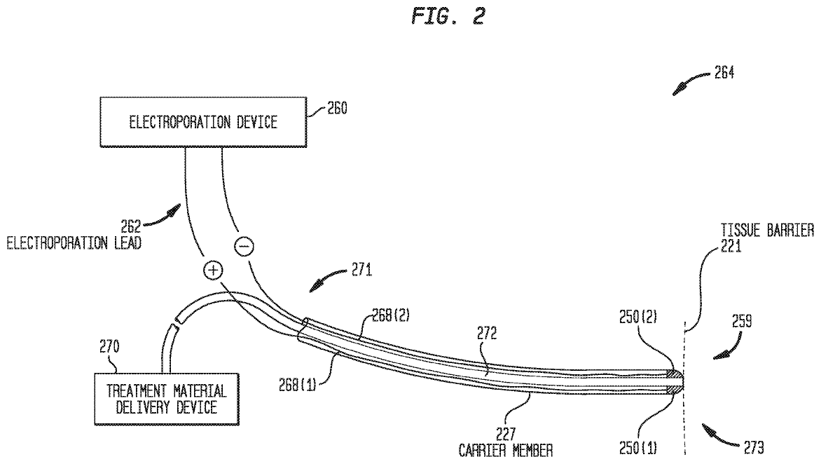

To this end, presented herein are techniques to introduce therapeutic substances into a sealed body chamber via electroporation gene therapy. Tissue barrier electroporation comprises introduction of genetic treatment material in the proximity of the tissue barrier cells to be electroporated before (prior to) or simultaneously with delivery of the electroporation electrical field. As such, in the embodiments of FIG. 2, the tissue barrier electroporation system 264 comprises a genetic treatment material delivery device 270 and a cannula 272 within the carrier member 227. In these embodiments, a solution containing the genetic treatment material to be transferred into the cells of the tissue barrier 221 is pumped into the spatial region proximate to the tissue barrier (e.g., into the middle ear). More specifically, genetic treatment material delivery device 270 is fluidically coupled to a proximal end 271 of the elongate cannula 272 extending longitudinally through the carrier member 227. The cannula 272 has a distal opening 273 located at the distal end 259 of the carrier member 227 (e.g., for supplying genetic treatment material to be transferred into the tissue barrier cells).

More specifically, in accordance with embodiments presented, a genetic treatment material is introduced proximate to a tissue barrier (e.g., the blood-labyrinth barrier) sealing a body chamber in a recipient. The cells of the tissue barrier are electroporated in order to transfer a least a portion of the treatment material into the cells of the tissue barrier. The genetic treatment material carries genetic code configured to make cells of the tissue barrier express (e.g., build or produce and release) one or many therapeutic substances (e.g., therapeutic proteins) into the sealed body chamber. As such, the therapeutic substances are introduced into the body chamber without the risk of side effects associated direct delivery (e.g., without compromising the integrity of the tissue barrier, without mechanical damage to the structures within the body chamber, etc.) or the risk of side effects associated systemically circulating therapeutic substances (e.g., high dosage).

FIG. 2 is a schematic diagram of a tissue barrier electroporation system 264, in accordance with certain embodiments presented herein. As shown, the tissue barrier electroporation system 264 comprises a carrier member 227 in/on which two electroporation electrode contacts (electrodes) 250(1) and 250(2) are positioned/disposed. In the specific example of FIG. 2, the electroporation electrodes 250(1) and 250(2) are positioned at a distal end (tip) 259 of the carrier member 227. As described further below, the carrier member 227 is configured for introduction of genetic treatment material in proximity to a tissue barrier 221.

The electroporation electrodes 250(1) and 250(2) are electrically connected to an external or implanted electroporation device 260, which is configured to generate electroporation signals (current signals) to be delivered via the electroporation electrodes 250(1) and 250(2). More specifically, in this example, carrier member 227 includes two isolated electrical conductors (e.g. wires) 268(1) and 268(2), each of which terminate at one of the electroporation electrodes 250(1) and 250(2), respectively. The electrical conductors 268(1) and 268(2) extend through the electroporation lead 262 to the electroporation device 260. As shown in FIG. 2, the electroporation electrodes 250(1) and 250(2) are positioned in proximity to, adjacent to, or abutting, the tissue barrier 221 (e.g., blood-labyrinth barrier).

The genetic treatment material delivery device 270 may be electrically operated (e.g., by a pump, pressurized reservoir, etc.) or may be manually operated (e.g., a syringe, manual pump, etc.) to supply a solution comprising the treatment material to be transferred into the tissue barrier cells. The flow rate of the fluid delivery device may be set at a constant or variable flow rate to ensure an effective amount of the genetic treatment material is delivered to the appropriate location for electroporation.

As noted above, in accordance with the tissue barrier electroporation techniques presented herein, an electroporation electrical field is generated via (by) the electroporation electrodes 250(1) and 250(2) proximate to the target tissue barrier 221 within the body of the recipient. During electroporation, the electroporation signals may be delivered repeatedly, periodically, etc., via the electroporation electrodes 250(1) and 250(2).

As noted, electroporation refers to the application of an electrical field to a cell in a manner that creates an electrical potential (i.e., voltage difference) across the cell membrane that, in turn, temporarily opens pores in the lipid bilayer membrane of the cell. In accordance with embodiments presented herein, the electrically opened pores in a tissue barrier, such as tissue barrier 221, are used to allow the genetic treatment material, which is charged to move within the electrical field, to enter the interior of the cells of the tissue barrier through the cell membrane (i.e., as the potential difference is applied to the cell, the electrically opened pores in the cell membrane allow the genetic treatment material to flow into the cell). After the electrical potential is removed, the pores in the cell membrane close such that the genetic treatment material remains in the cells of the tissue barrier 221.

As noted above, in the embodiments of FIGS. 2A-2C, two electroporation electrodes 250(1) and 250(2) are positioned at the distal end (tip) 259 of the carrier member 227. It is to be appreciated that the use of two electroporation electrodes is merely illustrative and that, in other embodiments, four, six, eight, ten, or more electrodes may positioned in, on, or near the carrier member 227 for use during electroporation. In other embodiment, at least one electroporation electrode may positioned in, on, or near the carrier member 227 and in electrical contact to the tissue barrier and at least a second electrode is positioned in some distance to the carrier member 227 and in electrical contact with the same tissue barrier to act as return electrode.

In certain embodiments, a number of electroporation electrodes can be arranged as multiple different anode/cathode pairs. The use of multiple different anode/cathode pairs can enable the delivery of different electroporation pulses at each pair. It might be beneficial to apply pulses with difference attributes, such as with increasing voltage or amplitude, changing polarity, etc. so that the cells are subjected to a loci of increasing (or decreasing) field strength and changing direction. Such changes in field direction may be beneficial to the electroporation process to, for example, increase the amount of electroporated cells. Alternating the voltage amplitude and polarity of the electroporation pulses could also be implemented with a single electrode pair, as shown in FIG. 2.

As noted, in the embodiments of FIGS. 2A-2C, a genetic treatment material is introduced proximate to the tissue barrier 221 (e.g., blood-labyrinth barrier) in a recipient and the cells of the tissue barrier 221 are electroporated to transfer a least a portion of the genetic treatment material into the cells of blood-labyrinth barrier. In accordance with certain embodiments presented herein, the genetic treatment material includes one or more nucleic acids, such as one or more of deoxyribonucleic acid (DNA) or Ribonucleic acid (RNA) vectors encoding therapeutic proteins. In certain embodiments, the DNA or RNA vectors encoding therapeutic proteins are referred to as being “naked” because it is isolated form any protein or other organic structure that might protect it (as is the case when contained in a chromosome, for example) and is therefore expressed in the cells.

Naked DNA or RNA vectors used to transfect a cell can be in the form of a plasmid, for example. As such, in certain embodiments, the genetic treatment material may be referred to as one or more “naked DNA plasmids encoding therapeutic proteins” and/or “naked RNA plasmids encoding therapeutic proteins.” For example, naked DNA plasmids encoding therapeutic proteins are small, circular, double stranded DNA that carry the genetic code; target tissue barrier cells that receive DNA plasmids may express one or many therapeutic substances in the form of “therapeutic proteins” or “protein-based therapeutics” that function inside the target tissue barrier cell.

The non-viral Ribonucleic acid (RNA) vectors or non-viral Deoxyribonucleic acid (DNA) (e.g., naked DNA plasmids encoding therapeutic proteins) encodes a gene sequence that encodes a sequence of amino acids, which a tissue barrier cell can build and put together into a long molecule. Any such molecule longer than twenty (20) amino acids is referred to as a “protein.” For building such proteins, there are twenty (20) types of amino acids available as building blocks, including nine (9) essential amino acids: isoleucine, leucine, lysine, methionine, phenylalanine, threonine, tryptophan, and valine; six (6) conditionally essential amino acids: arginine, cysteine, glycine, glutamine, proline, and tyrosine; and five (5)|dispensable amino acids: alanine, aspartic acid, asparagine, glutamic acid and serine. Depending on the source, human cells produce between 80,000 and 400,000 different types of proteins. Other types of genetic treatment material may also include, for example, other types of nucleic acids such as miRNA, shRNA, siRNA, and others, which can be used to precisely block the production of certain proteins in the tissue barrier.

In certain examples, the non-viral vectors can include other types of plasmids or naked DNA; Oligonucleotides; Antibiotic resistance free miniplasmids, such as operator repressor titration plasmids (pORTs), plasmids with conditional origin of replication pCORs), or plasmids free of antibiotic resistance (pFAR, e.g., pFAR4-CMVp-BDNF-IRES-NT3); Circular Covalently Closed Vectors, such as Minicircle, Minivector, or Miniknot); Linear Covalently Closed Vectors (“dumbbell-shaped”), such as minimalistic immunologically defined gene expression (MIDGE), micro-linear vectors (MiLVs), Ministring); Mini-intronic plasmids, etc.

In accordance with certain embodiments presented herein, the non-viral DNA vector causes the tissue barrier cells to express the therapeutic proteins (protein-based therapeutics) into the body chamber. The therapeutic proteins can include, for example, including peptides, recombinant proteins, monoclonal antibodies and vaccines, antibody-based drugs, Fc fusion proteins, anticoagulants, blood factors, bone morphogenetic proteins, engineered protein scaffolds, enzymes, growth factors, hormones, interferons, interleukins, and thrombolytics. In certain embodiments, the therapeutic proteins are neurotrophins, a family of proteins (hormones), including a brain-derived neurotrophic factor (BDNF), Neurotrophin 3 (NT3), Neurotrophin 4 (NT4), Nerve growth factor (NGF) and the endogenous steroids, dihydroepiandosterone (DHEA) and dihydroepiandosterone sulfate (DHEA-S). In one specific embodiment, the non-viral Deoxyribonucleic acid (DNA) vector encoding a therapeutic protein is pFAR4-CMVp-BDNF-IRES-NT3, which results in the release of the therapeutic proteins BDNF and NT3 to be released in the environment of the transfected target cells. The pFAR4-CMVp-BDNF-IRES-N is one specific example of an antibiotic resistant miniplasmid plasmid Free of Antibiotic Resistance markers version 4 (pFAR4) encoding BDNF and NT3, where pFAR4 is the plasmid backbone. The plasmid has a gene sequence encoding Brian-Derived Neurotrophic Factor (BDNF), Neurotrophin-3 (NT3), and a Cytomegalovirus promoter (CMVp). In accordance with other embodiments presented herein, the genetic treatment material may comprise other nucleic acids such as, for example, siRNA, shRNA and miRNA.

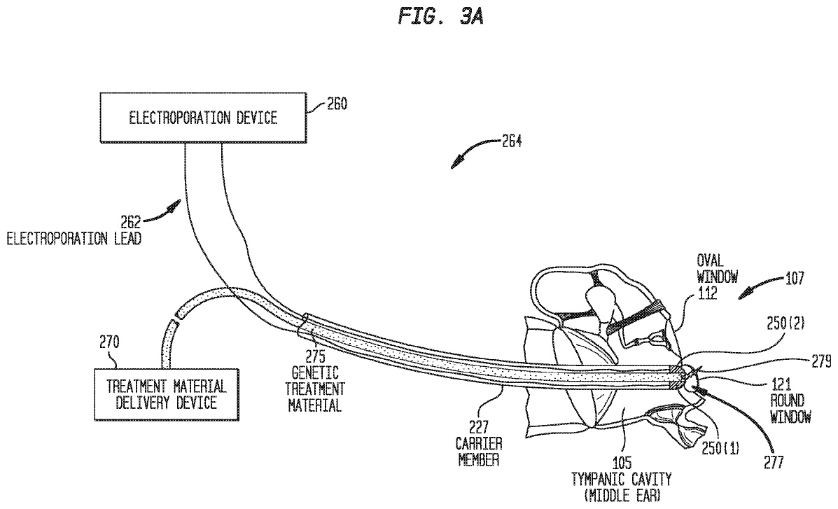

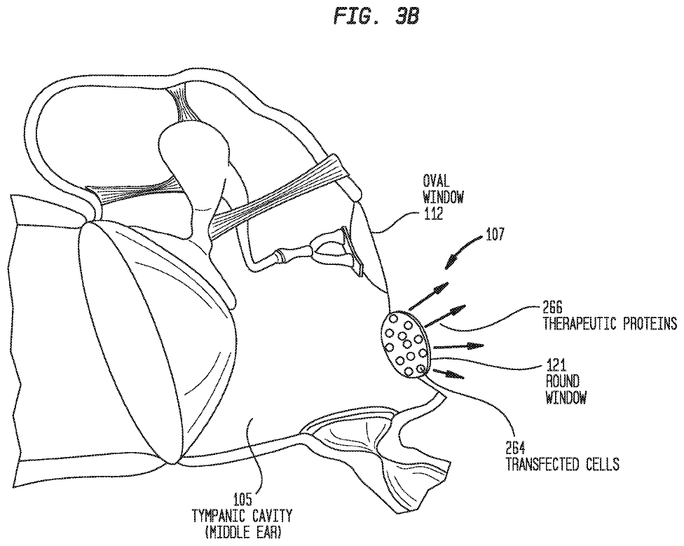

FIGS. 3A and 3B are simplified schematic diagram specifically illustrating electroporation gene therapy of a recipient's blood-labyrinth barrier, namely at the round window. For ease of description, the embodiments of FIGS. 3A and 3B will be described with reference to inner ear 107 of FIG. 1 and the electroporation system 264 of FIG. 2.

More specifically, as shown in FIG. 3A, the carrier member 227 is inserted into the recipient such that the two electroporation electrodes 250(1) and 250(2) pass through middle ear 105 and are positioned in proximity to (e.g., abutting) the round window 121. Also as shown in FIG. 3A, a genetic treatment material 275 is introduced into the spatial region 277 proximate to the round window 121 via cannula 272. Once the two electroporation electrodes 250(1) and 250(2) are positioned, and the genetic treatment material 275 is introduced into the spatial region 277, the round window 121 is electroporated to open pores in the round window cells. That is, the two electroporation electrodes 250(1) and 250(2) deliver electroporation signals 279 to the cells of the round window 121 in order to enable transfer of the treatment material 275 into those cells. Tissue barrier cells that have been electroporated, and in which a genetic treatment material has been transferred, are sometimes referred to herein as “transfected” tissue barrier cells.

In general, the electroporation signals 279 comprise current waveforms of sufficient magnitude/amplitude and duration to cause pores to form in the cell membranes. Waveform shapes that may be used include, but are not limited to, square pulses, exponentially decaying pulses, step pulses, ramped pulses, alternating polarity, etc. The electroporation signals 279 may comprise, for example, monopolar pulses, biphasic pulses, etc. In addition, an electroporation signal 279 can be formed by any suitable number of pulses, including two, three, four, five, six, seven, eight, nine, ten, fifteen, twenty or more pulses. However, by minimizing the number of electroporation electrodes, damage to cells from high fields may also be minimized. With fewer electrodes, and by dynamically moving the electrode along a path, a smaller field may be generated and electroporation may still be performed with high efficiency. That is, in contrast to the use of one large field to perform electroporation (as is the case in conventional static arrangements), dynamic electroporation uses a series of smaller fields during travel/movement of the carrier member and, as a result, cells proximate to the electroporation electrodes may experience fewer damaging conditions during electroporation (when compared to damage occurring with the use of one large field to perform electroporation).

The attributes of the electroporation signals 279 may vary depending upon the cell type undergoing electroporation. For example, electroporation may necessitate higher voltages, different pulse rates, etc. than for other applications, such as neurostimulation of the spinal cord. In certain aspects, lower currents may be used for electroporation, as cells may be subjected to repeated pulses as the electroporation electrode is moved along a path adjacent to the cells. In general, the round window, or other tissue barrier electroporation, includes application of regular (e.g., continuous, periodic, etc.) current pulses to the cells of the round window 121 via the electroporation electrodes 250(1) and 250(2).

FIG. 3B schematically illustrates cells 264 of the round window 121 into which the genetic treatment material 275 has been transferred. As noted, these cells are sometimes referred to herein as “transfected tissue barrier cells” 264. FIG. 3B also schematically illustrates that the transfected tissue barrier cells 264, in response to the genetic treatment material 275, express (e.g., generate/produce and release) therapeutic substances, in the form of therapeutic proteins 266, into the inner ear 107. As a result, the therapeutic proteins 266 are introduced into the inner ear 107 without the risk of side effects associated direct delivery to the inner ear 107 (e.g., without compromising the integrity of the blood-labyrinth barrier, without mechanical damage to the structures of the inner ear etc.) or the risk of side effects associated systemically circulating therapeutic substances (e.g., high dosage).

In other words, FIGS. 3A and 3B illustrate techniques for applying electrical signals to the blood-labyrinth barrier (including, e.g., in the proximity of the cells of the round window membrane) to make the blood-labyrinth barrier cells more porous. A solution that includes genetic treatment material (e.g., non-viral Deoxyribonucleic acid (DNA) vector encoding a therapeutic protein) is present and the genetic treatment material enters the blood-labyrinth barrier cells. Stated differently, the blood-labyrinth barrier cells receive the foreign genetic treatment material (via the temporarily created openings), driven by a concentration gradient through these openings. The genetic treatment material is generally charged so that it will move in the electric field into the blood-labyrinth barrier cells (voltage field gradient to deliver sections of DNA into the cells). The blood-labyrinth barrier cells express (generate and release) the desired therapeutic substance (e.g., therapeutic protein) in cells of the inner ear. As such, the transfected blood-labyrinth barrier cells (blood-labyrinth barrier cells into which the genetic treatment material has been transferred via electroporation) function as a therapeutic factory, without damaging, e.g., the round window membrane, in the process of delivering a therapeutic agent to the cells of the blood labyrinth barrier and inner ear.

In particular, the genetic treatment material varies, and can comprise different types of non-viral Deoxyribonucleic acid (DNA) vectors or other nucleic acids and other types of molecules as discussed herein, introduced simultaneously or sequentially into the proximity of a tissue barrier prior to and/or during electroporation. Depending on the condition to be treated, the introduction of different types of genetic treatment material in proximity to a tissue barrier may enable targeting of the same type or different tissue barrier cells for expression (or blocking of expression of) different therapeutic proteins into the cells of the inner ear, beyond the tissue barrier. As a result, the same or different tissue barrier cells can express different therapeutic proteins into the body chamber behind the tissue barrier.

FIGS. 2, 3A, and 3B illustrate embodiments in which genetic treatment material 275 is introduced via a cannula 272 within the carrier member 227. In such embodiments, the genetic treatment material 275 can be, for example, suspended or dissolved in any suitable liquid, e.g., water, saline, gel, etc. to form a solution. In general, the solution will have a level of conductivity suitable for performing electroporation. The genetic treatment material 275 may be in any suitable media or form, for example, liquid, gel, solid or particle.

The specific arrangement for introduction of the genetic treatment material shown in FIGS. 2, 3A, and 3B is illustrative of the introduction of solutions containing such material into the spatial region 277 (or other region adjacent to a target tissue barrier) using any suitable device (e.g., needle, syringe, cannula, catheter, capillary tube, etc.). Solutions may be injected simultaneously with, before, or after inserting or retracting the carrier member 227. In other embodiments, genetic treatment material are delivered via a delivery device that is separate from the electroporation electrodes, released from the carrier member 227 (e.g., elution from silicone rubber, etc.), released from an excipient immobilized on or near the carrier member 227 such as from hydrogels (e.g., Poloxamer (Pluronic), hyaluronic acid (e.g., Healon), etc.), film forming systems (e.g., Unisun's PVA with nanoparticles, etc.), biodegradable matrices (e.g., PLGA, etc.), pellets (e.g., O-Ray, etc.), nanoparticles, etc.

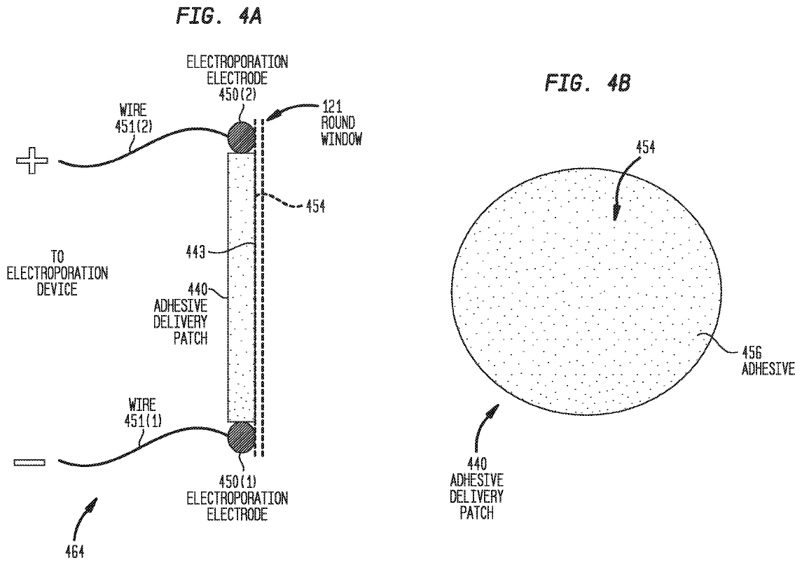

FIGS. 4A and 4B illustrate an alternative embodiment using an electroporation system 464 and a separate therapeutic delivery device in the form of an adhesive delivery patch 440 for introduction of a genetic treatment material FIG. 4A illustrates the electroporation system 464 and the adhesive delivery patch 440 in use with round window 121 from FIG. 1, while FIG. 4B illustrates the adhesive delivery patch 440 separate from the round window 121 and from the electroporation system 464. For ease of description, FIGS. 4A and 4B will be described together.

The electroporation system 464 comprises two electroporation electrodes 450(1) and 450(2) and an electroporation device (not shown in FIG. 4A). As shown in FIG. 4A, the two electroporation electrodes 450(1) and 450(2) are positioned proximate to the round window 121 and are each electrically connected to the electroporation device via respective wires 451(1) and 451(2). Similar to the above embodiments, the electroporation device is configured to use the electroporation electrodes 450(1) and 450(2) to electroporate the cells of the round window 121.

Also shown in FIG. 4A is the adhesive delivery patch 440. In these embodiments, the adhesive delivery patch 440 comprises a first surface 454 that is configured to be adhered to an outside/proximal surface 443 of the round window 121. That is, in use, the first surface 454 is configured to be positioned in proximity to, immediately adjacent to, or even abutting the outside/proximal surface 443 of the round window 121. Moreover, the adhesive delivery patch 440 is resiliently flexible and includes an adhesive 456 disposed in or on the first surface 454. The adhesive 456 is configured to adhere the first surface 454 to the proximal surface 443 of the round window 121.

In certain embodiments, the adhesive 456 comprises an adhesive gel or adhesive film disposed on the first surface 454. In other embodiments, the adhesive 456 is integrated into the first surface 454 (e.g., the first surface is formed so as to have adhesive properties). In such embodiments in which the adhesive 456 is integrated into the first surface 454, a covering can be provided on the first surface 454 prior to implantation of the adhesive delivery patch 440 into a recipient. In any event, the adhesive 454 ensures that the adhesive delivery patch 440 remains abutting the round window 121 at least during electroporation.

As noted above, a purpose of the adhesive delivery patch 440 is to deliver/introduce genetic treatment material into a proximity of the round window 121. Therefore, in the example of FIGS. 4A and 4B, the adhesive delivery patch 440 can be formed from, or include one or more genetic treatment materials. In certain embodiments, one or more genetic treatment materials are integrated into the adhesive delivery patch 440 and are released via, for example, elution, resorption, etc. For example, the adhesive delivery patch 440 can be formed using a scaffold or crystalline structure where the scaffold/crystal structure is loaded/doped with one or more therapeutic substances. In other embodiments, the adhesive delivery patch 440 can be coated with one or more genetic treatment materials.

In one illustrative arrangement, the adhesive delivery patch 440 can include a plurality of genetic treatment materials. In such embodiments, the genetic treatment materials can be configured to cause the round window 121 to, following electroporation, express proteins with different therapeutic effects, different release profiles (e.g., different release timelines), and/or other differences. In certain embodiments, the adhesive delivery patch 440 is fully bioresorbable, meaning the adhesive delivery patch 440 will, over time, be fully resorbed by the body.

Again, it is to be appreciated that the use of an adhesive delivery patch, such as shown in FIGS. 4A and 4B, is merely illustrative one of the many techniques that can be used to introduce genetic treatment materials in proximity to the blood-labyrinth barrier or other tissue barrier. For example, in one alternative embodiment, the adhesive delivery patch could be replaced with a hydrogel, fast forming agent, etc., loaded with one or more genetic treatment materials.

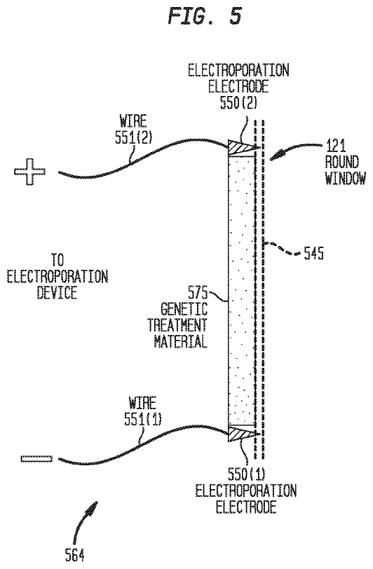

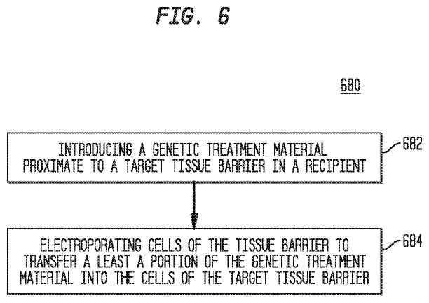

The above embodiments have generally been described with reference to electroporation electrodes positioned at or in proximity to an outer/proximal surface of a tissue barrier. FIG. 5 illustrates an alternative embodiment in which the electroporation electrodes are configured to only partially penetrate a tissue barrier.

More specifically, FIG. 5 illustrate an electroporation system 564 with separately introduced genetic treatment material 575. FIG. 5 illustrates the electroporation system 564 and the genetic treatment material 575 in use with round window 121 from FIG. 1.

The electroporation system 64 comprises two electroporation electrodes 550(1) and 550(2) and an electroporation device (not shown in FIG. 5). As shown in FIG. 4A, the two electroporation electrodes 550(1) and 550(2) each have a thin elongate shape with distal points 553(1) and 553(2), respectively. As such, the two electroporation electrodes 550(1) and 550(2) are sometimes referred to herein as “penetrating electrodes.”

In these arrangements, the penetrating electrodes 550(1) and 550(2) are configured to be positioned such that the distal points 553(1) and 553(2) only partially penetrate the round window 121. Similar to the above embodiments, the electroporation device is configured to use the penetrating electrodes 450(1) and 450(2) to electroporate the cells of the round window 121. The penetration of the round window 121 via the penetrating electrodes 450(1) and 450(2) may, following electroporation, enable the genetic treatment material 575 to reach more round window cells and/or cells on the perilymph/distal facing surface 545 of the round window 121.

In general, the penetrating electrodes are biocompatible (e.g., made from or coated with platinum, gold, graphene, Platinum iridium). To increase charge transfer capacity, the electrodes can have a modified surface, such as to include high-charge transfer coatings. These modified surfaces can include, for example, conductive polymers and hydrogels, Iridium oxides, highly roughened surface patterns, etc.

FIG. 6 is flowchart illustrating an example method 680, in accordance with embodiments presented herein. Method 680 begins at 682 where a genetic treatment material is introduced into a body of a recipient proximate to a target tissue barrier in the body of the recipient. In certain embodiments, the target tissue barrier is the blood-labyrinth barrier. At 684, cells of the target tissue barrier are electroporated to transfer a least a portion of the genetic treatment material into the cells of the tissue barrier.

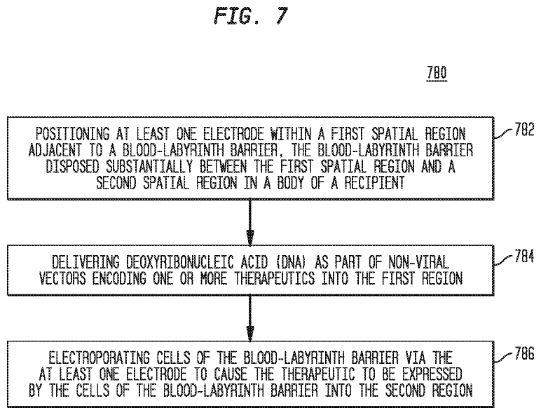

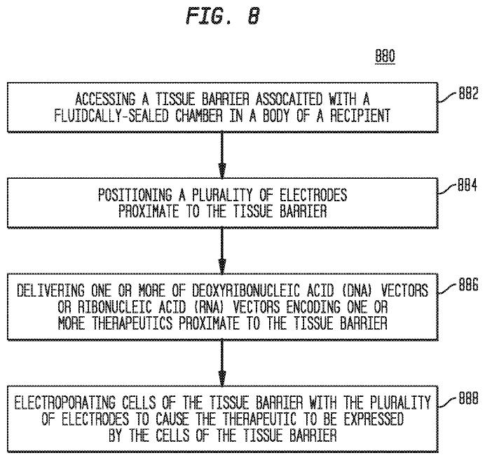

FIG. 7 is flowchart illustrating an example method 780, in accordance with embodiments presented herein. Method 780 begins at 782 where a surgeon positions at least one electrode within a first spatial region adjacent to a blood-labyrinth barrier, where the blood-labyrinth barrier is disposed substantially between the first spatial region and a second spatial region in a body of a recipient. At 784, the surgeon delivers non-viral deoxyribonucleic acid (DNA) vectors encoding one or more therapeutics into the first spatial region. At 786, the surgeon electroporates cells of the blood-labyrinth barrier via the at least one electrode to cause the therapeutic to be expressed by the cells of blood-labyrinth barrier cells into the second spatial region.

FIG. 8 is flowchart illustrating an example method 880, in accordance with embodiments presented herein. Method 880 begins at 882 where a surgeon accesses a tissue barrier associated with a fluidically-sealed chamber in a body of a recipient. At 884, the surgeon positions a plurality of electrodes proximate to the tissue barrier. At 886, the surgeon delivers non-viral deoxyribonucleic acid (DNA) vectors encoding one or more therapeutics proximate to the tissue barrier. At 888, the surgeon electroporates cells of the tissue barrier with the plurality of electrodes to cause the therapeutic to be expressed by (or to block the expression of certain proteins in) the cells of the tissue barrier and the inner ear.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A method, comprising:
introducing genetic treatment material proximate to a blood-labyrinth barrier in a recipient; and
electroporating cells of the blood-labyrinth barrier to transfer at least a portion of the genetic treatment material into the cells of the blood-labyrinth barrier.

2. The method of claim 1, wherein introducing the genetic treatment material comprises introducing a genetic treatment material configured to alter a biological composition of the cells of the blood-labyrinth barrier.

3. The method of claim 1, wherein introducing the genetic treatment material comprises introducing a nucleic acid into the cells of the blood-labyrinth barrier.

4. The method of claim 3, wherein introducing the nucleic acid comprises introducing one or more of deoxyribonucleic acid (DNA) vectors or Ribonucleic acid (RNA) vectors into the cells of the blood-labyrinth barrier.

5. The method of claim 4, wherein introducing one or more of deoxyribonucleic acid (DNA) vectors or Ribonucleic acid (RNA) vectors into the cells of the blood-labyrinth barrier comprises:
introducing one or more of non-viral DNA vectors encoding at least one therapeutic protein or non-viral RNA vectors encoding at least one therapeutic protein into the cells of the blood-labyrinth barrier.

6. The method of claim 5, wherein the at least one therapeutic protein comprises:
at least one neurotrophin.

7. The method of claim 6, wherein the at least one neurotrophin comprises:
at least one brain-derived neurotrophic factor (BDNF).

8. (canceled)

9. The method of claim 5, wherein the one or more of non-viral DNA vectors encoding at least one therapeutic protein or non-viral RNA vectors encoding at least one therapeutic protein comprise:
at least one naked DNA vector.

10. The method of claim 5, wherein the one or more of non-viral DNA vectors encoding at least one therapeutic protein or non-viral RNA vectors encoding at least one therapeutic protein comprise:
at least one plasmid.

11. The method of claim 4, wherein introducing one or more of non-viral DNA vectors encoding at least one therapeutic protein or non-viral RNA vectors encoding at least one therapeutic protein comprises:
introducing one or more of non-viral DNA vectors or one or more non-viral RNA vectors each encoding two or more therapeutic proteins.

12. The method of claim 1, wherein electroporating the cells of the blood-labyrinth barrier comprises:
placing at least one active electrode adjacent to, and in electrical contact with, a proximal surface of the blood-labyrinth barrier without penetrating the blood-labyrinth barrier;
placing at least one return electrode in electrical contact to the proximal surface of the blood-labyrinth barrier; and
generating an electroporation electric field at the cells of the blood-labyrinth barrier via the at least one active electrode and at least one return electrode.

13. The method of claim 1, wherein electroporating the cells of the blood-labyrinth barrier comprises:
inserting at least one penetrating electrode into the recipient, wherein the at least one penetrating electrode only partially penetrates the blood-labyrinth barrier; and
generating an electroporation electric field at the cells of the blood-labyrinth barrier via the at least one penetrating electrode and one or more other electrodes.

14. (canceled)

15. The method of claim 1, wherein electroporating the cells of the blood-labyrinth barrier comprises:
electroporating at least one of a round window membrane or an oval window membrane of an inner ear of the recipient.

16. (canceled)

17. The method of claim 1, wherein introducing the genetic treatment material proximate to the blood-labyrinth barrier comprises:
delivering a solution comprising the genetic treatment material to a spatial region proximate to the blood-labyrinth barrier.

18. (canceled)

19. The method of claim 1, wherein introducing the genetic treatment material proximate to the blood-labyrinth barrier comprises:
adhering an adhesive patch to the blood-labyrinth barrier, wherein the adhesive patch is loaded with the genetic treatment material.

20. The method of claim 1, wherein introducing the genetic treatment material proximate to the blood-labyrinth barrier comprises:
positioning a hydrogel adjacent to the blood-labyrinth barrier, wherein the hydrogel is loaded with the genetic treatment material.

21. The method of claim 1, wherein the genetic treatment material comprises charged molecules configured to move in a presence of an electrical field.

22. The method of claim 1, wherein introducing the nucleic acid into the cells of the blood-labyrinth barrier comprises:
introducing any one or more of shRNA, siRNA, or miRNA molecules suitable to block expression of one or more proteins.

23. The method of claim 1, wherein the nucleic acid introduced into the cells of the blood-labyrinth barrier comprises:
delivering the nucleic acid as one component of a conjugated molecule.

24. The method of claim 1, further comprising:
positioning at least one electrode within a first spatial region adjacent to the blood-labyrinth barrier, the blood-labyrinth barrier disposed substantially between the first spatial region and a second spatial region in a body of a recipient; and
electroporating cells of the blood-labyrinth barrier via the at least one electrode to cause the genetic treatment material to be expressed by the cells of the blood-labyrinth barrier into the second spatial region.

25.-44. (canceled)
